(19) United States
(12) Patent Application Publication
(10) Pub. No.: US 2022/0213047 A1
(43) Pub. Date: Jul. 7, 2022

(54) CARBONIC ANHYDRASE INHIBITORS FOR TREATMENT OF NEISSERIA GONORRHOEAE INFECTION

(71) Applicant: Purdue Research Foundation, West Lafayette, IN (US)

(72) Inventors: Daniel P. Flaherty, West Lafayette, IN (US); Mohamed Seleem, Blacksburg, VA (US)

(73) Assignee: Purdue Research Foundation, West Lafayette, IN (US)

(21) Appl. No.: 17/552,413

(22) Filed: Dec. 16, 2021

Related U.S. Application Data
(60) Provisional application No. 63/133,256, filed on Jan. 1, 2021.

(51) Int. Cl. C07D 285/135; A61P 31/04; C07D 417/12

(52) U.S. Cl. CPC C07D 285/135; A61K 45/06; C07D 417/12; A61P 31/04

(57) ABSTRACT

The invention described generally relates to novel therapeutic compounds, and in particular to carbonic anhydrase inhibitors as an antibiotic against Neisseria gonorrhea bacteria and methods for treating those sexually transmitted infection diseases in mammals using the described carbonic anhydrase inhibitors having a formula (I),

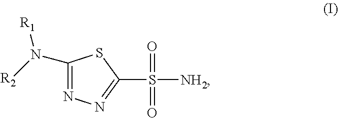

or a pharmaceutical formulation thereof, alone or together with one or more other antibiotics.

### CROSS-REFERENCE TO RELATED APPLICATIONS

The present U.S. patent application relates to and claims the priority benefit of U.S. Provisional Patent Application Ser. No. 63/133,256, filed Jan. 1, 2021, the contents of which are hereby incorporated by reference in its entirety into this disclosure.

### TECHNICAL FIELD

The present disclosure generally relates to novel therapeutic compounds, and in particular to carbonic anhydrase inhibitors for treatment of Neisseria gonorrhea infections and methods for treating infection diseases caused by Neisseria gonorrhea using the described carbonic anhydrase inhibitors or a pharmaceutical formulation thereof.

### BACKGROUND

This section introduces aspects that may help facilitate a better understanding of the disclosure. Accordingly, these statements are to be read in this light and are not to be understood as admissions about what is or is not prior art.

Gonorrhea is a sexually transmitted disease caused by the bacterial pathogen Neisseria gonorrhoeae that colonizes urogenital, anal, and nasopharyngeal tissues. The World Health Organization (WHO) estimated there were 78 million new cases of gonorrhea in adults worldwide in 2008¹. In the United States specifically, the Centers for Disease Control and Prevention (CDC) reported a 67% increase in gonorrhea cases between 2013-2017 with >550,000 cases reported in 2017 alone. However, this is believed to be a gross underestimation as gonorrhea can present as both symptomatic and asymptomatic. It is estimated that asymptomatic colonization makes up more than half of the infected individuals at any one time, and it is this version that greatly promotes transmission of the pathogen³. Both versions wreak havoc on world health care systems causing pelvic inflammatory disease, infertility and ectopic pregnancies⁴. The bacteria can also be transmitted from mother to child during birth and lead to blindness⁵. If left untreated, N. gonorrhoeae can cause gonococcemia resulting in skin infection, arthritis or endocarditis^{6,7}.

Pathogenic gonorrhea strains are increasingly resistant to common front-line antibiotics. The WHO surveillance program reports resistance to available antibiotics including □-lactams, tetracycline and quinolines⁸ leaving the only options for treatment being a combination of azithromycin and third-generation cephalosporins, to which resistance has been documented as well. Rampant resistance has caused the CDC and the WHO each to classify N. gonorrhoeae as a superbug⁹ and a future with an untreatable gonococcal infection is a real possibility¹⁰. The CDC has listed drug-resistant N. gonorrhoeae at the highest possible threat level to public health¹¹, and the WHO has called for an international collaborative effort to combat the infection¹². This highlights the significant unmet need to identify novel targets and molecules with therapeutic potential. Efforts to develop a vaccine toward N. gonorrhoeae are still in the discovery phase with questions remaining regarding and the effectiveness of immune response in mucosal membranes¹³. Furthermore, existing antibiotics such as delafloxacin and the clinical molecule solithromycin investigated against gonorrhea failed in clinical trials as they did not meet the criteria for non-inferiority relative to current treatment options. Altogether, this highlights the limited number of antibacterials in clinical development (and they inhibit limited number of molecular targets) which further emphasizes the unmet needs to discover new antibacterial agents for gonorrhea.

### BRIEF DESCRIPTION OF DRAWINGS

FIG. 1. Structure of FDA-approved carbonic anhydrase (CA) Acetazolamide (AZM).

FIG. 2. Structure of FDA-approved carbonic anhydrase (CA) Ethoxzolamide (EZM).

FIG. 3A. Normalized fluorescence of tracer 13 with dose-response of NgCA to determine K_{d} of tracer. FIG. 3B. Competition assay with constant tracer and NgCA concentration and dose-response of AZM to determine Kᵢ.

FIG. 4. Study and analog design feedback loop.

FIG. 5. AZM works synergistically with AZI and inhibits growth of N. gonorrhoeae.

FIG. 6. Multi-step resistance selection.

FIG. 7. Intracellular clearance of N. gonorrhoeae.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Unless defined otherwise, all technical and scientific terms used herein have the same meaning as commonly understood to one of ordinary skill in the art. As defined herein, the following terms and phrases shall have the meanings set forth below.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, Or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

In some illustrative embodiments, the invention is related to a method for treating a patient with a bacterial infection comprising the step of administrating therapeutically effective amount of a carbonic anhydrase inhibitor or a pharmaceutically acceptable salt thereof, together with one or more diluents, excipients or carriers, to the patient in need of treatment for said infection.

In some illustrative embodiments, the invention is related to a method for treating a patient with a bacterial infection comprising the step of administrating therapeutically effective amount of a carbonic anhydrase inhibitor or a pharmaceutically acceptable salt thereof, used in combination with one or more other antibiotics, together with one or more diluents, excipients or carriers, to the patient in need of treatment for said infection.

In some illustrative embodiments, the invention is related to a method for treating a patient with a bacterial infection comprising the step of administrating therapeutically effective amount of a carbonic anhydrase inhibitor or a pharmaceutically acceptable salt thereof, together with one or more diluents, excipients or carriers, to the patient in need of treatment, wherein said bacterial is Neisseria gonorrhea bacteria.

In some illustrative embodiments, the invention is related to a method for treating a patient with a bacterial infection comprising the step of administrating therapeutically effective amount of a carbonic anhydrase inhibitor or a pharmaceutically acceptable salt thereof, used in combination with one or more other antibiotics, together with one or more diluents, excipients or carriers, to the patient in need of treatment for said infection, wherein said bacterial is Neisseria gonorrhea bacteria.

In some illustrative embodiments, the invention is related to a method for treating a patient with a bacterial infection comprising the step of administrating therapeutically effective amount of a carbonic anhydrase inhibitor or a pharmaceutically acceptable salt thereof, used in combination with one or more other antibiotics, together with one or more diluents, excipients or carriers, to the patient in need of treatment for said infection, wherein said infection disease is a sexually transmitted infection disease.

In some illustrative embodiments, the invention is related to a method for treating a patient with a bacterial infection comprising the step of administrating therapeutically effective amount of a carbonic anhydrase inhibitor or a pharmaceutically acceptable salt thereof, together with one or more diluents, excipients or carriers, to the patient in need of treatment for said infection, wherein said carbonic anhydrase inhibitor has the formula (I)

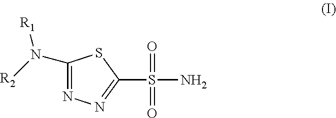

or a pharmaceutically acceptable salt thereof, wherein
R₁ is hydrogen, an acyl, alkyl, alkenyl, alkynyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted; and
R₂ is hydrogen, an acyl, alkyl, alkenyl, alkynyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted.

In some illustrative embodiments, the invention is related to a compound for treating a patient with a bacterial infection comprising the step of administrating therapeutically effective amount of a carbonic anhydrase inhibitor or a pharmaceutically acceptable salt thereof, together with one or more diluents, excipients or carriers, to the patient in need of treatment, wherein said carbonic anhydrase inhibitor has the formula (I)

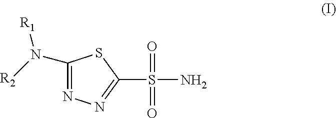

or a pharmaceutically acceptable salt thereof, wherein
R₁ is hydrogen, an acyl, alkyl, alkenyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted; and
R₂ is hydrogen, an acyl, alkyl, alkenyl, alkynyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted.

In some illustrative embodiments, the invention is related to a method for decolonizing a patient with a bacterial infection comprising the step of administrating therapeutically effective amount of a carbonic anhydrase inhibitor or a pharmaceutically acceptable salt thereof, together with one or more diluents, excipients or carriers, to the patient in need of treatment, wherein said carbonic anhydrase inhibitor

In some illustrative embodiments, the invention is related to a composition for treating a patient with a symptom caused a bacterial infection comprising the step of administrating therapeutically effective amount of a carbonic anhydrase inhibitor or a pharmaceutically acceptable salt thereof, together with one or more diluents, excipients or carriers, to the patient in need of treatment, wherein said symptom is an inflammatory bowel disease or autoimmune disease, wherein said carbonic anhydrase inhibitor has the formula (I)

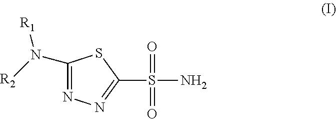

or a pharmaceutically acceptable salt thereof, wherein
R₁ is hydrogen, an acyl, alkyl, alkenyl, alkynyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted; and
R₂ is hydrogen, an acyl, alkyl, alkenyl, alkynyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted.

In some illustrative embodiments, the invention is related to a method for treating a patient with a symptom caused a bacterial infection comprising the step of administrating therapeutically effective amount of a carbonic anhydrase inhibitor or a pharmaceutically acceptable salt thereof, together with one or more diluents, excipients or carriers, to the patient in need of treatment, wherein said symptom is an inflammatory bowel disease or autoimmune disease, wherein said carbonic anhydrase inhibitor is

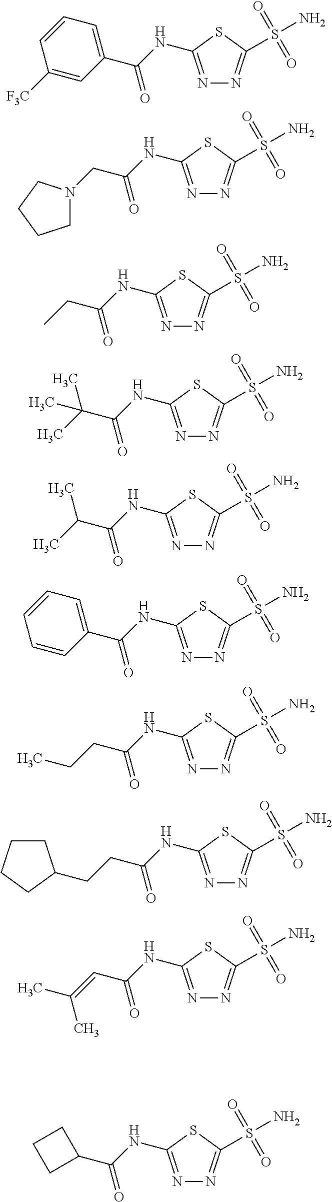

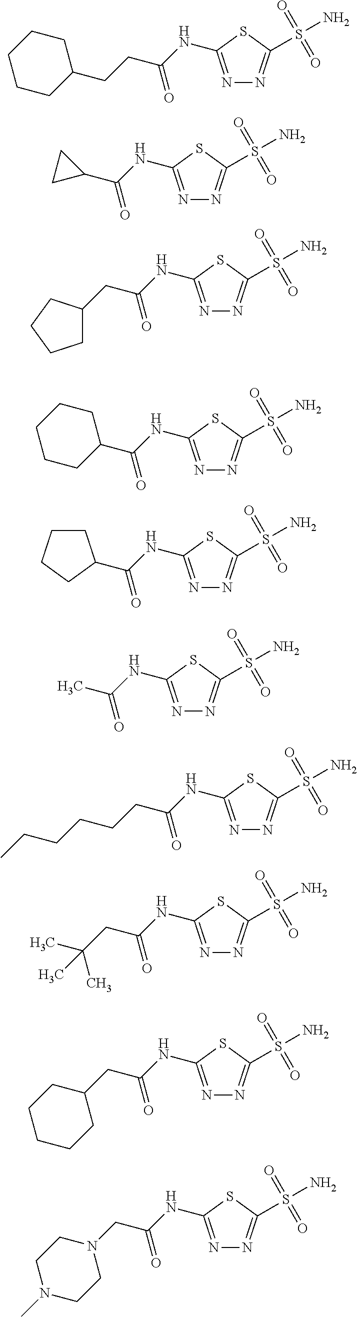

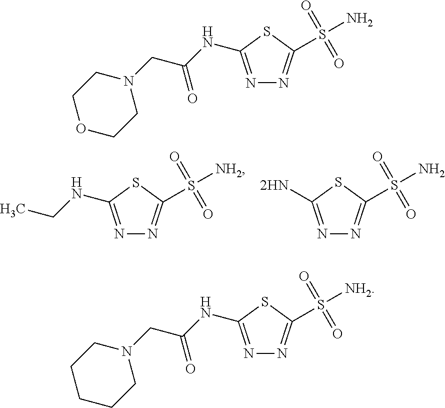

In some illustrative embodiments, the invention relates to a method for treating a patient with a bacterial infection comprising the step of administrating therapeutically effective amount of a carbonic anhydrase inhibitor or a pharmaceutically acceptable salt thereof, together with one or more diluents, excipients or carriers, to the patient in need of treatment for said infection.

In some other illustrative embodiments, the present invention relates to a pharmaceutical composition comprising one or more compounds of formula (I) as disclosed herein, or a pharmaceutically acceptable salt thereof, together with one or more diluents, excipients or carriers.

In some other illustrative embodiments, the present invention relates to a pharmaceutical composition comprising one or more compounds of formula (I) as disclosed herein, or a pharmaceutically acceptable salt thereof, together with one or more diluents, excipients or carriers, for use as a medicament.

In some other illustrative embodiments, the present invention relates to a pharmaceutical composition comprising one or more compounds of formula (I) as disclosed herein, or a pharmaceutically acceptable salt thereof, together with one or more diluents, excipients or carriers, for use in the treatment of a bacterial infection.

In some other illustrative embodiments, the present invention relates to a method for treating a patient with a bacterial infection comprising the step of administrating therapeutically effective amount of a carbonic anhydrase inhibitor or a pharmaceutically acceptable salt thereof, in combination with one or more other compounds of the same or different mode of action, together with one or more diluents, excipients or carriers, to the patient in need of treatment for said infection.

In another embodiment, pharmaceutical compositions containing one or more of the compounds are also described herein. In one aspect, the compositions include a therapeutically effective amount of the one or more compounds for treating a patient with infection. It is to be understood that the compositions may include other component and/or ingredients, including, but not limited to, other therapeutically active compounds with the same or different modes of action, and one or more carriers, diluents, excipients, and the like. In another embodiment, methods for using the compounds and pharmaceutical compositions for treating patients with infection are also described herein. In one aspect, the methods include the step of administering one or more of the compounds and/or compositions described herein to a patient with infection. In another aspect, the methods include administering a therapeutically effective amount of the one or more compounds and/or compositions described herein for treating patients with infection.

The following non-limiting exemplary embodiments are included herein to further illustrate the invention. These exemplary embodiments are not intended and should not be interpreted to limit the scope of the invention in any way. It is also to be understood that numerous variations of these exemplary embodiments are contemplated herein.

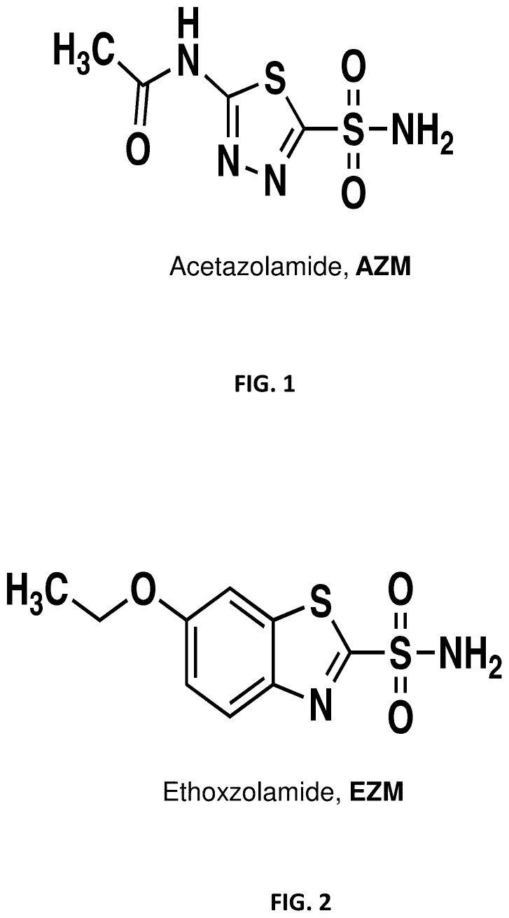

Carbonic anhydrases (CAs) are a group of zinc-metalloenzymes that consist of α, β, and γ sub-groups found in all kingdoms of life. CAs catalyze the essential reaction of converting carbon dioxide (CO₂) and water to bicarbonate, HCO₃⁻, and a proton¹⁴. In humans there are 16 α-CA isoforms with broad tissue distribution that carry out the reaction. The reaction is relevant in many physiological processes such as transport of CO₂ from metabolizing tissues to excretion in the lungs¹⁵, maintaining pH and CO₂ homeostasis in various tissues¹⁶, and regulating electrolyte secretion in various tissues and organs¹⁷⁻¹⁹. These roles have made CAs prime drug targets as FDA approved carbonic anhydrase inhibitors (CAI) are used to treat various disorders including glaucoma^{20,21,} as a diuretic for kidney health²², treatment for congestive heart failure²³, and recently certain isoforms have gained momentum as promising cancer targets²⁴⁻²⁶. Aside from human targets, CAs have been identified in a handful of parasites and pathogenic bacteria²⁷, including N. gonorrhoeae. In bacteria, altering bicarbonate homeostasis was revealed to perturb the proton motive force and reduce bacterial fitness²⁸. It was first observed in 1967 that N. gonorrhoeae had limited susceptibility to the FDA approved CAI acetazolamide (AZM, FIG. 1)²⁹. Thirty years later the N. gonorrhoeae CA (NgCA) was first cloned³⁰ and a crystal structure bound to AZM was published a year later³¹. NgCA was further characterized enzymatically³² and genomic data finally identified NgCA as an essential enzyme in N. gonorrhoeae in 2014³³. NgCA is classified as an □-CA residing in the periplasmic space and is required for maintaining CO₂ and pH homeostasis for the organism and has been suggested to be a valuable new drug target to treat gonorrhea²⁷. However, until now, there has never been a concerted effort to target NgCA as a means to combat this pathogen.

Repurposing already approved drugs with well-characterized toxicology and pharmacology is an attractive way to reduce the time, cost and risk associated with antibiotic innovation³⁴⁻³⁶. Studies proposed in this application build upon discoveries that FDA-approved CAIs and novel analogs developed by our team, exhibit potent, narrow-spectrum, and on-target antimicrobial activity in an applicable clinical range, against highly multidrug-resistant N. gonorrhoeae isolates. We hypothesize that NgCA is a valid anti-gonococcal drug target and that CAIs represent a novel and effective therapeutic option for treating N. gonorrhoeae. The scientific premise for this hypothesis is based on the supporting literature described above and our preliminary studies. AZM, in particular, is an attractive candidate to repurpose for several reasons: 1) it has an ideal safety profile as up to 1-gram dose/day can be given with no toxicity observed in humans³⁷, 2) it exhibits ideal pharmacokinetics (PK, good renal clearance and no metabolites)³⁸, 3) low treatment cost. For these combined reasons, AZM is listed as an essential medicine by the WHO³⁹. Moreover, there is increased interest in repurposing CAIs for other medical conditions due to their well-established safety/PK profiles illustrated by 79 clinical studies currently underway for acetazolamide alone (ClinicalTrials.gov).

We have demonstrated the potential of AZM, ethoxzolamide (EZM, FIG. 2), and novel analogs, to be used in the treatment of gonococcal infection (preliminary study). Early structure-activity relationship (SAR) studies have improved the potency from MIC₅₀=4 μg/mL for AZM to 0.5 μg/mL for our most potent analogs while EZM displayed an initial potency of MIC₅₀=0.125 μg/mL. In addition, we have demonstrated on-target binding to NgCA and have an in vitro binding assay to characterize analogs. We have also successfully crystallized NgCA in our laboratory which further support the feasibility of a structure-based design approach. Finally, we provide evidence that one analog may inhibit more than one target in N. gonorrhoeae reducing the chance of resistance. And have yet to isolate resistance mutants toward either CAI. Importantly, AZM selectivity inhibits N. gonorrhoeae over commensal bacteria that protect against pathogenic colonization of the gut and vaginal lining.

First, repurposing drugs, with well-characterized toxicology and pharmacology, to find new applications outside the scope of the original medical indication is a novel way to reduce both the time and cost associated with antimicrobial innovation³⁶. Second, the successful demonstration that a non-antimicrobial, such as AZM, is capable of (just to mention a few): 1) selectively killing N. gonorrhoeae species, 2) not harming normal gut or vaginal microbiota, 3) inhibiting N. gonorrhoeae growth at a clinically achievable dose, 4) exhibiting a long post-antibiotic effect, 5) avoiding rapid resistance development, 6) working synergistically with approved antibiotics, 7) demonstrating intracellular efficacy in clearing N. gonorrhoeae infected cervical cells comparable to the current drugs of choice all increase the level of innovation for this proposal. Adding to the innovative nature of the proposal is that we have evidence that certain analogs not just engage the desired target, NgCA, but also exhibit their antibacterial effects by inhibiting at least one other target. This polypharmacology reduces the potential for N. gonorrhoeae to develop resistance to the AZM scaffold.

This innovative approach also highlights a unique target identified for these drugs that has never been explored before for antimicrobial drug discovery, and could ultimately be exploited in future screening campaigns for new inhibitory scaffolds.

An additional innovative aspect of our proposed study is that we have improved the potency of the scaffold versus N. gonorrhoeae by approximately 8-fold compared to AZM without the assistance of a structure-based approach. We posit the inclusion of rational design against NgCA, and any potential secondary targets, would greatly enhance the effectiveness of future analogs.

The proposed research is expected to have a significant positive impact on human health by validating analogs of the FDA approved drug AZM as potent and effective anti-gonococcal agents. This may lead to the development of a new class of antibacterials in a fraction of the time and at a significantly lower cost than is required for traditional antimicrobial discovery routes. It is clear that these drugs have the potential to make a safe, effective, quick, novel and affordable impact on treatment of gonorrhea.

Carbonic Anhydrase Inhibitors Display Efficacy Against N. gonorrhoeae

The FDA-approved CAI AZM was initially tested for anti-gonococcal activity, against a set of N. gonorrhoeae clinical isolates according to Clinical & Laboratory Standards Institute (CLSI) guidelines⁴⁰. These tests were performed in normal culture conditions with standard CO₂ levels and in CO₂ (5% in incubator) conditions. With CO₂ being the natural substrate for CAs, it will compete against inhibitors that are on-target for NgCA resulting in reduced susceptibility for N. gonorrhoeae to the molecules. After initial testing of AZM and analogs we evaluated other FDA-approved CAIs and EZM proved to be superior. Both were tested by agar dilution on a larger panel of N. gonorrhoeae strains (39 strains, Table 1). AZM and EZM display MIC₅₀ values of 4 and 0.125 μg/mL in standard CO₂ conditions (MIC₉₀'s of 16 and 0.5 μg/mL), respectively, while the molecules showed significant reduction of activity under CO₂ conditions. This data indicates the molecules are exhibiting their antimicrobial activity via inhibition of NgCA.

Carbonic Anhydrase Inhibitors Display Narrow-Spectrum Activity

We have shown that CAIs display a narrow-spectrum of activity (Table 6). We have previously shown that CAIs have potential as anti-enterococcal agents for decolonization of the gut and treating systemic vancomycin-resistant enterococci (VRE) infections without affecting commensal strains common in the human microbiome or other pathogenic bacteria. However, Enterococcus species do not encode for carbonic anhydrases; therefore, the anti-enterococcal activity is not due to inhibiting carbonic anhydrase but a yet to be characterized novel target (on-going research). It is important to note that the design of anti-enterococcal agents, as part of a newly funded NIAID proposal, does not overlap in scope or aim with this current proposal because while the initial “hit” molecules are the same starting points for optimization the intracellular targets, approach, and SAR diverge significantly. The extremely narrow spectrum of this class of inhibitors would be a positive for clinical use as the molecules: 1) would not have deleterious effects on normal microbiota, and 2) would have less opportunity for resistance to arise to other important pathogenic strains aside from VRE.

AZM Binds Directly to NgCA

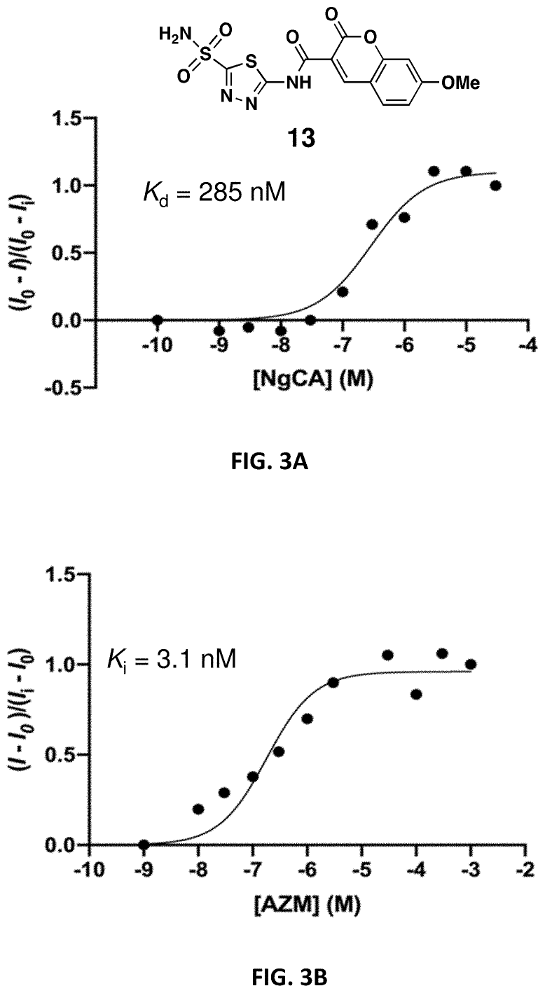

Measuring the inhibition of the reaction catalyzed by CAs requires an expensive stopped-flow apparatus and intricate assay design to monitor CO₂ consumption in a matter of micro-seconds⁵¹. Alternatively, a method was published recently using of an AZM-based fluorophore probe (13, FIG. 3A) to monitor direct binding to CAs. This assay was validated against several human CA isozymes⁵². The fluorescence of probe 13 is quenched by 90% when fully bound to CAs and once displaced in a competition binding assay will emit fluorescence at 420 nm. The K_{d} of the tracer 13 was measured by maintaining 500 nM concentration and titrating NgCA (0-30 μM) while reading fluorescence intensity. The normalized intensity was plotted as a function of NgCA concentration and fit with non-linear regression using the one-site specific inhibitor model in Prism to provide a K_{d} value of 285 nM (FIG. 3A) compared to 65 nM for the same tracer against hCAII⁵².

Next, the tracer and NgCA concentrations were kept constant at 300 nM each then subjected to competition with dose-response of analogs. The normalized intensity was plotted as a function of analog concentration and fitted with the Fit Kᵢ model in Prism to calculate a Kᵢ for representative analogs (FIG. 3B). A representative plot is shown for AZM which exhibits a Kᵢ=3.1 nM when plotted and adjusted for the K_{d} of the tracer. This is comparable to AZM versus many isoforms of hCAs where Kᵢ's range from 2.5 nM-74 nM¹⁴. This value is also comparable to the Kᵢ=1.0 nM for AZM determined using this same tracer against bovine CA in the original publication⁵². These data demonstrate the feasibility of the assay to accurately measure Kᵢ for the molecules versus NgCA.

Experimental Design: Optimization of AZM and EZM Analogs

While we have demonstrated with the current set of analogs based on AZM that we can develop molecules to engage NgCA and possess clinically relevant levels of potency against N. gonorrhoeae. However, there is still opportunity for improvement. Structure-based and traditional ligand designs are carried out in an iterative process with the goals to: 1) improve binding affinity toward NgCA, 2) improve selectivity versus human CAs, 3) achieve MIC₅₀'s<0.125 μg/mL, 4) maintain low frequency of resistance, 5) maintain selectivity over commensal microbiota, 6) maintain no toxicity versus human cell lines, and 7) maintain desirable physicochemical properties. After extensive rounds of rational design, we will arrive at 4-8 molecules for in vitro PK/ADMET profiling and evaluation in in vivo N. gonorrhoeae model.

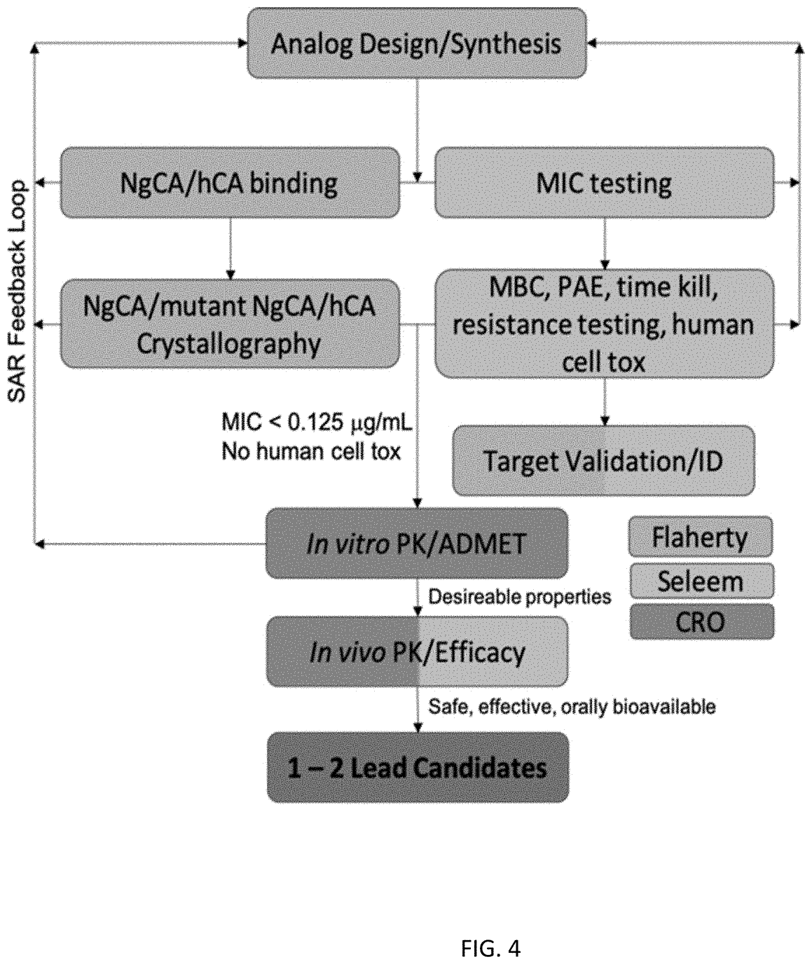

The experimental flowchart is presented in FIG. 4. Analogs are designed and synthesized in cohorts of 8-10 molecules. All analogs are tested in the following assays: 1) binding affinity versus NgCA and hCAs I-VI using the technique described in the feasibility section above, 2) in vitro antibacterial assays against N. gonorrhoeae strains and native human gut and urogenital tract microbes in normal and CO₂ conditions and 3) toxicity to human cell lines. All data are used to inform the next round of analog design in an iterative process. As part of our analog design protocol, we will use the cheminformatics software (Chem3D) to predict three-dimensional geometry of designed analogs to ensure molecules remain in the rod-to-disc like chemical space. Additionally, we will use bioinformatics software (pkCSM⁵³) to computationally predict properties associated with absorption, distribution, metabolism, excretion and toxicity (ADMET). SAR data are tracked using the program Canvas (Drug Discovery Suite, Schrödinger, LLC) to identify antibacterial trends pertaining to structural and physicochemical properties and develop SAR for future design.

TABLE 1
MIC of N. gonorrhoeae against CAI compounds in Non CO2 environment (μg/mL)

| | | N. gonorrhoeae | N. gonorrhoeae | N. gonorrhoeae |
|---|---|---|---|---|
| | | 178 | 181 | 194 |
| CAI 009 | 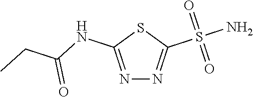 | 8 | 8 | 16 |
| CAI 0010 | 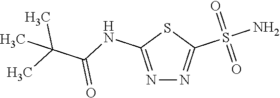 [SMILES: CC(C)(C)C(=O)Nc1nnc(S(N)(=O)=O)s1] | 32 | 32 | 32 |
| CAI 0011 | 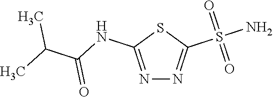 | 16 | 16 | 8 |
| CAI 0012 | 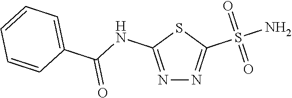 [SMILES: NS(=O)(=O)c1nnc(NC(=O)c2ccccc2)s1] | 2 | 4 | 2 |
| CAI 0013 | 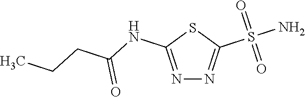 | 2 | 8 | 4 |
| CAI 0015 | 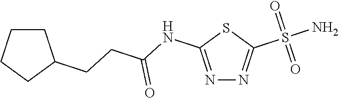 [SMILES: NS(=O)(=O)c1nnc(NC(=O)CCC2CCCC2)s1] | 2 | 1 | 4 |
| CAI 0016 | 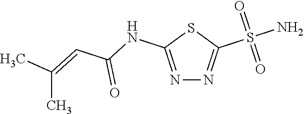 [SMILES: CC(C)=CC(=O)Nc1nnc(S(N)(=O)=O)s1] | 16 | 16 | 32 |
| CAI 0018 | 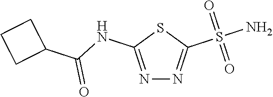 [SMILES: NS(=O)(=O)c1nnc(NC(=O)C2CCC2)s1] | 16 | 16 | 8 |
| CAI 0019 | 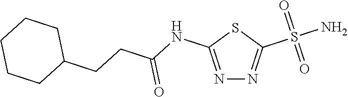 [SMILES: NS(=O)(=O)c1nnc(NC(=O)CCC2CCCCC2)s1] | 4 | 1 | 2 |
| CAI 0020 | 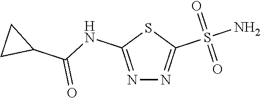 [SMILES: NS(=O)(=O)c1nnc(NC(=O)C2CC2)s1] | 2 | 2 | 4 |
| CAI 0021 | 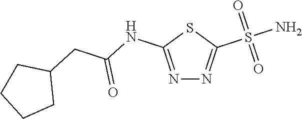 [SMILES: NS(=O)(=O)c1nnc(NC(=O)CC2CCCC2)s1] | 2 | 1 | 4 |
| CAI 0022 | 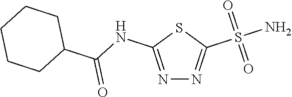 [SMILES: NS(=O)(=O)c1nnc(NC(=O)C2CCCCC2)s1] | 4 | 2 | 4 |
| CAI 0023 | 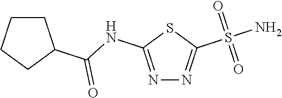 [SMILES: NS(=O)(=O)c1nnc(NC(=O)C2CCCC2)s1] | 8 | 8 | 8 |
| CAI 0026 | 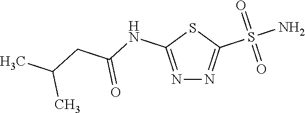 [SMILES: CC(C)CC(=O)Nc1nnc(S(N)(=O)=O)s1] | 8 | 8 | 4 |
| CAI 0027 | 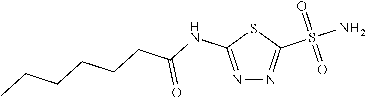 [SMILES: CCCCCCC(=O)Nc1nnc(S(N)(=O)=O)s1] | 2 | 2 | 1 |
| CAI 0028 | 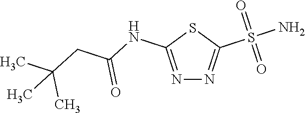 [SMILES: CC(C)(C)CC(=O)Nc1nnc(S(N)(=O)=O)s1] | 8 | 16 | 32 |
| CAI 0029 | 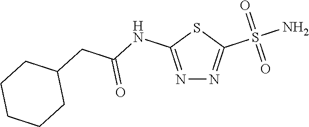 [SMILES: NS(=O)(=O)c1nnc(NC(=O)CC2CCCCC2)s1] | 1 | 0.5 | 2 |
| CAI 0030 | 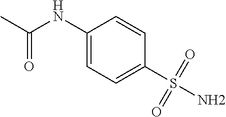 | 8 | 32 | 16 |
| CAI 0031 | 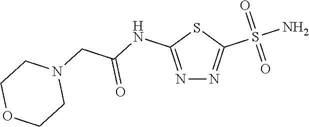 [SMILES: NS(=O)(=O)c1nnc(NC(=O)CN2CCOCC2)s1] | 32 | >64 | 64 |
| CAI 0032 | 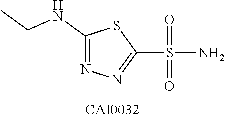 | 64 | >64 | >64 |
| CAI 0034 | 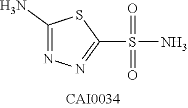 | 1 | 4 | 2 |
| CAI 0035 | 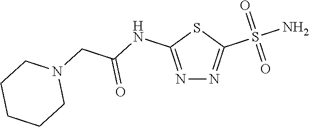 [SMILES: NS(=O)(=O)c1nnc(NC(=O)CN2CCCCC2)s1] | 64 | >64 | 64 |
| CAI 0036 | 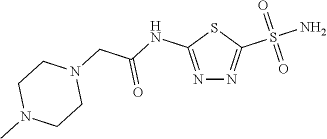 [SMILES: CN1CCN(CC(=O)Nc2nnc(S(N)(=O)=O)s2)CC1] | 64 | >64 | >64 |
| CAI 0037 | 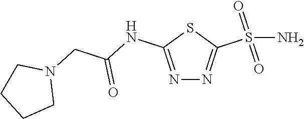 [SMILES: NS(=O)(=O)c1nnc(NC(=O)CN2CCCC2)s1] | >64 | >64 | >64 |
| CAI 0038 | 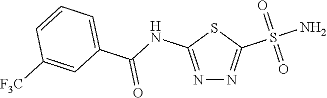 [SMILES: NS(=O)(=O)c1nnc(NC(=O)c2cccc(C(F)(F)F)c2)s1] | 1 | 4 | 2 |
| Azithromycin | Control | 0.25 | >128 | 0.25 |
| Ciprofloxacin | Control | 16 | ≤0.5 | ≤0.5 |

In Vitro Efficacy Versus N. gonorrhoeae and Pharmacokinetic/ADMET Evaluation.

To date, we have developed AZM analogs with improved potency, from 4 μg/mL to 0.5 μg/mL, versus a panel of N. gonorrhoeae strains. The anti-gonococcal activity is on target against NgCA, as the molecules either have reduced or no activity against N. gonorrhoeae in CO₂ conditions. However, an optimized analog still maintained appreciable activity against N. gonorrhoeae in CO₂ conditions a possible second target. In this aim, we will confirm/identify intracellular target(s) by the analogs tested and continue in vitro testing of analogs against various strains of N. gonorrhoeae and normal microbiota. We will also investigate possible mechanisms of resistance to help design next generation inhibitors that may overcome resistance.

Preliminary Data: New AZM Analog Exhibits Anti-Gonococcal Activity in CO₂ Conditions

If the antimicrobial activity of the molecules is a result of inhibiting only NgCA then the molecules should display a loss of activity in CO₂ conditions. This is indeed the case for AZM and EZM. However, some analogs did maintain weak potency against N. gonorrhoeae under CO₂ conditions indicating the possibility of a second target. For example, analog 10 exhibited an MIC=0.5 μg/mL in normal culture conditions and MIC of 4 μg/mL in CO₂ conditions. Moreover, our team has yet to isolate resistant mutants toward any of the analogs tested to date. These data indicate the possibility that analogs, particularly 10, may be engaging a second target. EZM did not maintain any activity against N. gonorrhoeae in CO₂ conditions. Hence, it is possible this attribute is exclusive to AZM based analogs.

TABLE 2
MIC of N. gonorrhoeae in normal growth
environment (with CO₂) (μg/mL)

| | N. gonorrhoeae | N. gonorrhoeae | N. gonorrhoeae |
|---|---|---|---|
| | 178 | 181 | 194 |
| CAI 009 | 16 | >64 | 64 |
| CAI 0010 | 32 | >64 | 64 |
| CAI 0011 | 32 | >64 | 64 |
| CAI 0012 | 16 | >64 | 64 |
| CAI 0013 | 8 | 32 | 8 |
| CAI 0015 | 16 | >64 | 64 |
| CAI 0016 | 64 | 32 | 64 |
| CAI 0018 | 64 | >64 | 32 |
| CAI 0019 | 64 | 64 | >64 |
| CAI 0020 | 64 | 64 | >64 |
| CAI 0021 | 64 | >64 | >64 |
| CAI 0022 | 32 | 64 | >64 |
| CAI 0023 | 32 | 64 | 64 |
| CAI 0026 | 32 | >64 | >64 |
| CAI 0027 | 32 | 64 | 64 |
| CAI 0028 | 32 | 64 | 64 |
| CAI 0029 | 32 | >64 | >64 |
| CAI 0030 | >64 | >64 | >64 |
| CAI 0031 | 64 | >64 | >64 |
| CAI 0032 | >64 | >64 | >64 |
| CAI 0033 | >64 | >64 | >64 |
| CAI 0034 | >64 | >64 | >64 |
| CAI 0035 | >64 | >64 | >64 |
| CAI 0036 | >64 | >64 | >64 |
| CAI 0037 | >64 | >64 | >64 |
| CAI 0038 | >64 | 64 | >64 |
| Azithromycin | 1 | >128 | 0.5 |
| Ciprofloxacin | 16 | ≤0.5 | ≤0.5 |

TABLE 3
MIC of N. gonorrhoeae against Acetazolamide and its analogues in non-
CO2 environment (μg/mL)

| | | N. gonorrhoeae | N. gonorrhoeae | N. gonorrhoeae |
|---|---|---|---|---|
| | | 178 | 181 | 194 |
| Acetazolamide | 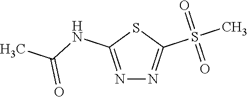 | 1 | 4 | 4 |
| Brinzolamide | 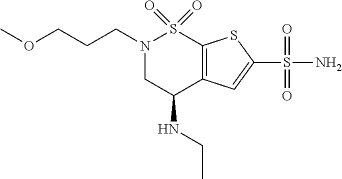 [SMILES: CCN[C@H]1CN(CCCOC)S(=O)(=O)c2sc(S(N)(=O)=O)cc21] | >64 | >64 | >64 |
| Dichlorphenamide | 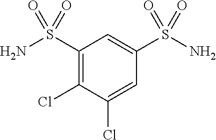 [SMILES: NS(=O)(=O)c1cc(Cl)c(Cl)c(S(N)(=O)=O)c1] | 2 | 8 | 4 |
| Dorzolamide | 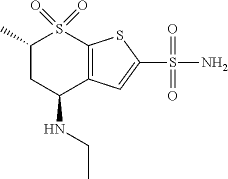 [SMILES: CCN[C@H]1C[C@H](C)S(=O)(=O)c2sc(S(N)(=O)=O)cc21] | 32 | >64 | >64 |
| Ethoxzolamide | 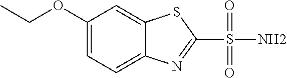 [SMILES: CCOc1ccc2nc(S(N)(=O)=O)sc2c1] | ≤0.5 | ≤0.5 | ≤0.5 |

TABLE 4
MIC of N. gonorrhoeae against Acetazolamide and its analogues
in normal growth environment (with CO₂) (μg/mL)

| | N. gonorrhoeae | N. gonorrhoeae | N. gonorrhoeae |
|---|---|---|---|
| | 178 | 181 | 194 |
| Acetazolamide | 32 | 64 | 64 |
| Brinzolamide | >64 | >64 | >64 |
| Dichlorphenamide | >64 | >64 | >64 |
| Dorzolamide | >64 | >64 | >64 |
| Ethoxzolamide | >64 | >64 | >64 |

TABLE 5
MICs against ATCC strain 700825

| Drug | Structure | ATCC Strain 700825 MIC (μg/mL) | |
|---|---|---|---|
| Acetazolamide | 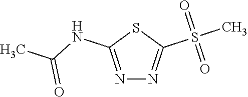 | In CO₂ 64 | In air 1 |
| Ethoxzolamide | 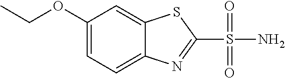 [SMILES: CCOc1ccc2nc(S(N)(=O)=O)sc2c1] | In CO₂ 64 | In air 0.5 |
| CAI 0019 | 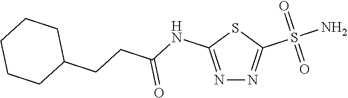 [SMILES: NS(=O)(=O)c1nnc(NC(=O)CCC2CCCCC2)s1] | In CO₂ 32 | In air 4 |
| CAI0031 | 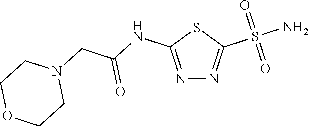 [SMILES: NS(=O)(=O)c1nnc(NC(=O)CN2CCOCC2)s1] | In CO₂ 128 | In air 32 |
| CAI 0040 | 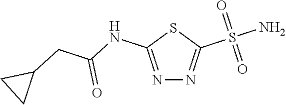 [SMILES: NS(=O)(=O)c1nnc(NC(=O)CC2CC2)s1] | In CO₂ 32 | In air 16 |
| CAI 0041 | 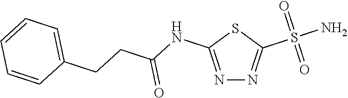 [SMILES: NS(=O)(=O)c1nnc(NC(=O)CCc2ccccc2)s1] | In CO₂ 4 | In air 0.5 |

TABLE 6
MIC50 for AZM and EZM against pathogenic
and commensal bacteria.

| | MIC₅₀ (μg/mL) | | | |
|---|---|---|---|---|
| Species | AZM | | EZM | |
| (# of strains) | normal | CO₂ | normal | CO₂ |
| N. gonorrhoeae (39) | 4 | 64 | 0.125 | >128 |
| Enterococcus (49) | 4 | nt | 4 | nt |
| Lactobacillus sp (12) | >256 | nt | >256 | nt |
| Bifidobacterium sp (18) | >256 | nt | >256 | nt |
| Bacteroides (9) | >256 | nt | >256 | nt |
| L. crispatus (2) | >256 | nt | >256 | nt |
| L. jensenii (3) | >256 | nt | >256 | nt |
| L. rhamnosus (1) | >256 | nt | >256 | nt |
| L. johnsonii (1) | >256 | nt | >256 | nt |
| L. gasseri (4) | >256 | nt | >256 | nt |
| E. coli (8) | >256 | nt | >256 | nt |
| S. aureus (12) | >256 | nt | >256 | nt |
| S. epidermidis (3) | >256 | nt | >256 | nt |
| S. pneumoniae (4) | >256 | nt | >512 | nt |
| E. coli TolC mutant (1) | >256 | nt | >256 | nt |
| Enterobacter spp. (4) | >256 | nt | >256 | nt |
| Klebsiella spp. (4) | >256 | nt | >256 | nt |

*“Normal” indicates standard conditions in ambient air. “CO₂” indicates bacteria was cultured in conditions containing 5% CO₂ in the incubator.
nt = not tested.

TABLE 7
Post-antibiotic Effect (PAE)

| | PAE (hours | | |
|---|---|---|---|
| Strains | AZM | EZM | AZI |
| Ng 181 | 2 | 10 | 8 |
| Ng 194 | 6 | 10 | 8 |
| Ng 186 | 4 | 10 | 8 |
| Ng 198 | 4 | 10 | 8 |

TABLE 8
Frequencies of Resistance
Frequency of Spontaneous Mutation (Original MIC)

| Strain | AZM | EZM | RIF |
|---|---|---|---|
| Ng 197 | <2.4 × 10⁻¹⁰ (1) | <2.4 × 10⁻¹⁰ (0.25) | 1.8 × 10⁻⁶ |
| Ng 202 | <2.4 × 10⁻¹⁰ (8) | <2.4 × 10⁻¹⁰ (0.06) | 4.2 × 10⁻⁶ |
| Ng 206 | <2.4 × 10⁻¹⁰ (4) | <2.4 × 10⁻¹⁰ (0.125) | 1.2 × 10⁻⁶ |

TABLE 9
MIC values for Acetazolamide and its two analogues
against 40 strains of N. gonorrhoeae in ambient air
and in 5% CO₂ containing air (μg/mL).

| | MIC values of the tested drugs (μg/mL) | | | | | |
|---|---|---|---|---|---|---|
| | Acetazolamide | | Dichlorphenamide | | Ethoxzolamide | |
| | | Non | | NON | | NON |
| Strains | CO₂ | CO₂ | CO₂ | CO₂ | CO₂ | CO₂ |
| N. gonorrhoeae | 64 | 2 | >128 | 2 | >128 | 0.125 |
| 165 | | | | | | |
| N. gonorrhoeae | 128 | 16 | >128 | 8 | >128 | 0.06 |
| 166 | | | | | | |
| N. gonorrhoeae | 128 | 2 | >128 | 4 | 64 | 0.25 |
| 167 | | | | | | |
| N. gonorrhoeae | 64 | 64 | >128 | 64 | >128 | 0.06 |
| 170 | | | | | | |
| N. gonorrhoeae | 32 | 2 | >128 | 1 | 64 | 0.125 |
| 171 | | | | | | |
| N. gonorrhoeae | 32 | 4 | >128 | 2 | 128 | 0.125 |
| 173 | | | | | | |
| N. gonorrhoeae | 64 | 1 | >128 | 2 | >128 | 0.125 |
| 174 | | | | | | |
| N. gonorrhoeae | >128 | 4 | >128 | 16 | >128 | 0.25 |
| 175 | | | | | | |
| N. gonorrhoeae | 64 | 1 | >128 | 4 | 128 | 0.25 |
| 176 | | | | | | |
| N. gonorrhoeae | 32 | 1 | 128 | 4 | 16 | 0.5 |
| 177 | | | | | | |
| N. gonorrhoeae | 128 | 1 | >128 | 1 | >128 | 0.06 |
| 178 | | | | | | |
| N. gonorrhoeae | 128 | 16 | >128 | 8 | >128 | 0.25 |
| 179 | | | | | | |
| N. gonorrhoeae | 32 | 4 | >128 | 8 | >128 | 0.125 |
| 180 | | | | | | |
| N. gonorrhoeae | 128 | 4 | 128 | 8 | 64 | 0.25 |
| 181 | | | | | | |
| N. gonorrhoeae | 16 | 1 | >128 | 16 | 64 | 0.25 |
| 182 | | | | | | |
| N. gonorrhoeae | 16 | 1 | 128 | 1 | 32 | 0.125 |
| 183 | | | | | | |
| N. gonorrhoeae | 16 | 4 | >128 | 4 | 32 | 0.25 |
| 184 | | | | | | |
| N. gonorrhoeae | 16 | 8 | >128 | 8 | 64 | 0.125 |
| 185 | | | | | | |
| N. gonorrhoeae | 16 | 2 | >128 | 8 | 64 | 0.125 |
| 186 | | | | | | |
| N. gonorrhoeae | 32 | 4 | >128 | 1 | 64 | 0.06 |
| 188 | | | | | | |
| N. gonorrhoeae | >128 | 4 | >128 | 4 | >128 | 0.06 |
| 191 | | | | | | |
| N. gonorrhoeae | 64 | 4 | 128 | 4 | 64 | 0.125 |
| 193 | | | | | | |
| N. gonorrhoeae | 64 | 4 | 128 | 4 | 64 | 0.25 |
| 194 | | | | | | |
| N. gonorrhoeae | 16 | 1 | >128 | 1 | >128 | 0.125 |
| 196 | | | | | | |
| N. gonorrhoeae | 16 | 1 | >128 | 4 | 128 | 0.25 |
| 197 | | | | | | |
| N. gonorrhoeae | 64 | 4 | >128 | 1 | >128 | 0.25 |
| 198 | | | | | | |
| N. gonorrhoeae | >128 | 16 | >128 | 32 | >128 | 0.25 |
| 200 | | | | | | |
| N. gonorrhoeae | >128 | 8 | >128 | 16 | 128 | 0.06 |
| 202 | | | | | | |
| N. gonorrhoeae | 128 | 1 | >128 | 1 | 64 | 0.125 |
| 203 | | | | | | |
| N. gonorrhoeae | >128 | 8 | >128 | 16 | 128 | 0.125 |
| 205 | | | | | | |
| N. gonorrhoeae | 128 | 4 | >128 | 4 | >128 | 0.125 |
| 206 | | | | | | |
| N. gonorrhoeae | 128 | 2 | >128 | 4 | 64 | 0.125 |
| 207 | | | | | | |
| N. gonorrhoeae | >128 | 8 | >128 | 4 | >128 | 0.125 |
| 208 | | | | | | |
| N. gonorrhoeae | >128 | 1 | >128 | 1 | >128 | 0.06 |
| 209 | | | | | | |
| N. gonorrhoeae | 64 | 1 | >128 | 4 | 64 | 0.125 |
| 210 | | | | | | |
| N. gonorrhoeae | 32 | 1 | >64 | 1 | 64 | 0.5 |
| 211 | | | | | | |
| N. gonorrhoeae | 32 | 16 | >128 | 32 | 64 | 0.25 |
| 212 | | | | | | |
| N. gonorrhoeae | 32 | 8 | >128 | 16 | 64 | 0.125 |
| 213 | | | | | | |
| N. gonorrhoeae | 32 | 1 | >128 | 1 | 64 | 0.25 |
| 214 | | | | | | |
| MIC 50 | 64 | 4 | >128 | 4 | >128 | 0.125 |
| MIC 90 | 64 | 16 | >128 | 16 | >128 | 0.5 |
| Modal MIC | 64 | 1 | >128 | 4 | >128 | 0.125 |

CAIs had no impact on vaginal microbiota—Vaginal microbiota compete with N. gonorrhoeae for adhesion to the urinary tract in addition to creating an acidic environment that prevent gonococcal colonization. Thus, we investigated the activity of CAIs against different species of Lactobacillus that comprise the female urogenital tract microbiota. CAIs exhibited strong selectivity towards N. gonorrhoeae without inhibiting growth of different Lactobacillus spp. In contrast, both drugs of choice for gonorrhea infections, azithromycin (AZI) and ceftriaxone, inhibited growth of the Lactobacillus spp. tested at concentrations below 1 μg/mL.

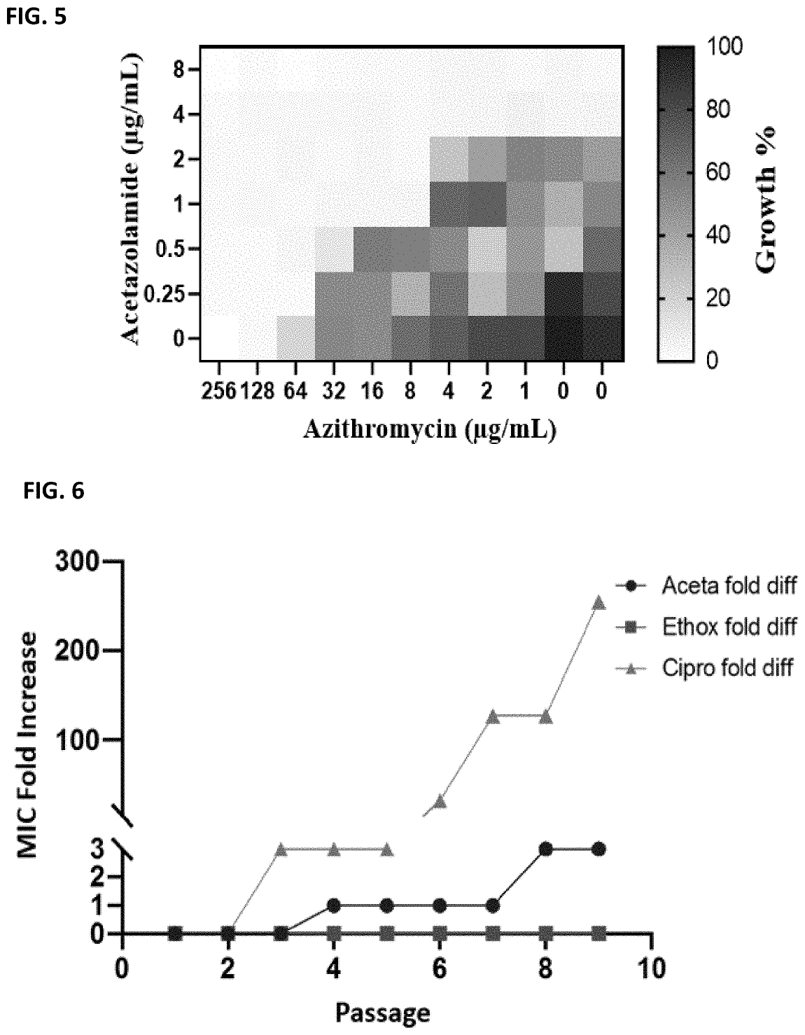

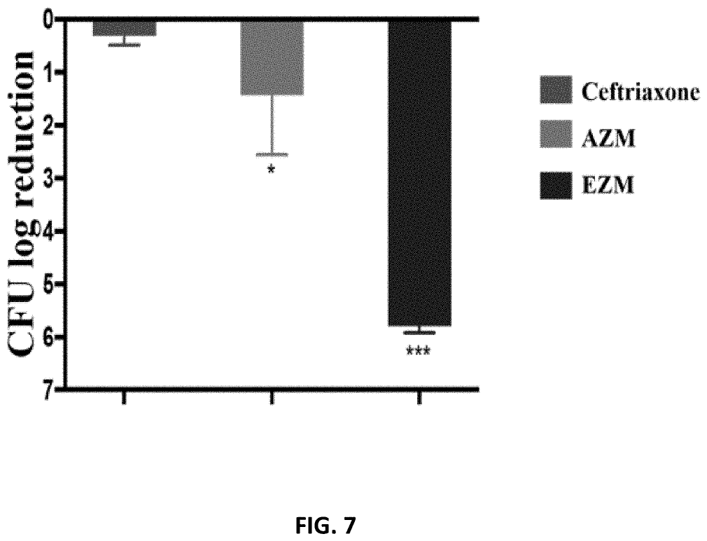

CAIs work synergistically with approved antibiotics—Dual therapy with ceftriaxone and azithromycin is the recommended approach to treat infections caused by N. gonorrhoeae. The use of two antibiotics in conjunction is thought to curb the rapid emergence of resistance to either antibiotic, if used alone. Thus, we investigated the potential of CAIs to be used in combination with other antibiotics against N. gonorrhoeae⁵⁶ (FIG. 5). Using a standard checkerboard assay, AZM and EZM were found to possess a synergetic relationship with AZI (AZM; FIC=0.125 and EZM; FIC=0.240). This suggests that dual therapy involving CAIs and azithromycin may be feasible, though further investigation is needed.

EZM exhibits a long post-antibiotic effect—In vitro pharmacodynamic analysis can provide valuable information regarding establishing a proper dosing regimen for drug candidates. One method to obtain this information is to determine if a compound/drug exhibits a post-antibiotic effect⁵⁷. The PAE for the CAIs, AZI was determined against clinical isolates of N. gonorrhoeae after exposure to 10×MIC for 1 hour. Table 7 reveals that all CAIs exhibit a long PAE ranging from 2-6 hours (for AZM) to 10 hours (for EZM). EZM was superior to what was observed with AZI (PAE 8 hours).

Frequency of mutation—single-step mutation—To assess the potential for rapid emergence of resistance of N. gonorrhoeae to CAIs, we attempted to generate a N. gonorrhoeae mutant that is resistant to CAIs using a single-step mutation assay⁵⁶. N. gonorrhoeae mutants exhibiting resistance to CAIs could not be isolated at 10× or 6×MIC (Table 8), indicating a low likelihood of rapid resistance emerging to these drugs (<2.4×10⁻¹⁰).

Frequency of mutation—multi-step mutation—We investigated N. gonorrhoeae's ability to develop resistance to CAIs using the multi-step resistance selection experiment⁵⁸. As depicted in FIG. 6, the MIC of AZM only increased two-fold over ten passages and no increase in the MIC for EZM was observed. In contrast, N. gonorrhoeae developed resistance rapidly to ciprofloxacin (Cipro) as a 300-fold increase in MIC was observed after 9 passages.

Intracellular clearance of N. gonorrhoeae—N. gonorrhoeae is known to invade epithelial cells of the genital tract and cross the epithelial barrier into the subepithelial space⁵⁹. Previous studies have shown that N. gonorrhoeae can survive inside host cells and pass epithelial cell layers (a key step in causing disseminated infections)^{60,61}. Therefore, a drug candidate must also be able to kill intracellular N. gonorrhoeae in order to be an effective therapeutic. Also, we were concerned that CAIs have reduced activity in high-CO2 conditions and may not be suitable to treat intracellular infection. Thus, we examined CAIs ability to reduce the burden of intracellular N. gonorrhoeae present in infected endocervical cells⁵⁶. Endocervical cells (End1/E6E7) were infected with N. gonorrhoeae and subsequently treated with either AZM, EZM or ceftriaxone (at 5×MIC) for 24 hours. AZM generated a 1.4-log₁₀ reduction in N. gonorrhoeae inside infected endocervical cells (97.7% reduction). Interestingly, EZM was superior to AZM and cleared N. gonorrhoeae (5.8 log₁₀ reduction) while ceftriaxone, in contrast, was ineffective (0.32-log₁₀ reduction) (FIG. 5). The results collectively indicate that CAIs have the ability to gain entry into endocervical cells and significantly reduce intracellular N. gonorrhoeae burden, at a rate that is superior to ceftriaxone.

Experimental Design:

In Vitro Assessment of AZM and EZM Antibacterial Properties

In addition to standard MIC assays molecules are evaluated for minimal bactericidal concentration (MBC), post-antibiotic effect (PAE) on the test strains, and time-kill curves. In addition, we will test the compounds ability to inhibit and eradicate intracellular N. gonorrhoeae and work synergistically with approved antibiotics. These are standard assays described previously by our group^{56,58,62,63}. The MIC and MBC are determined against ˜200 clinical isolates. Studies will include comparator antibiotics (azithromycin, ceftriaxone, and doxycycline) as controls.

MIC Against Normal Microflora

Rationale: Inhibition of the native intestinal microbiome leads to aggressive colonization of C. difficile and recurrence. Thus, we will test optimized compounds against key gut microflora to establish whether these compounds are still selective toward N. gonorrhoeae over normal gut microflora. Also, vaginal microbiota competes with N. gonorrhoeae for adhesion to the urinary tract in addition to creating an acidic environment that prevent gonococcal colonization. Thus, we will investigate the activity of CAIs against species of Lactobacillus that comprise the female urogenital tract microbiota.

Assess the Frequency of Resistance of Potent Leads

Although we were not able to isolate mutants to CAIs, the in vitro frequency of resistance studies of prioritized lead compounds are assessed to confirm it has been maintained a frequency of resistance <1×10⁻¹⁰ and that no clinical liabilities are associated with the mechanism(s) of resistance. Also, understanding the potential resistance mechanisms that can be avoided at the compound optimization stage is an essential step in drug development research.

Established techniques^{56,64-66} are used for evaluating frequency of resistance against N. gonorrhea.

Mutants of N. gonorrhea resistant to CAI derivatives are generated in vitro by two methods: (a) large volume of logarithmic culture of N. gonorrhea 10¹⁰-10¹¹ CFU/mL are plated on agar containing 2×, 4×, 10×, and 20×MIC compound⁶⁷⁻⁷⁰ and (b) using an alternative approach, mutants developed by CAI derivatives are isolated by multiple passage methods through progressively increasing concentrations of a compound in liquid culture^{71,72}. N. gonorrhoeae cultures that grew at the highest concentrations of the compound are used as an inoculum for the subsequent culture. Colonies from methods (a) and (b) are selected and mutants stable to a given compound are confirmed^{69,70}.

Genomic DNA are isolated from single colonies using standard methods⁷³. Bar coded indexed sequencing libraries are constructed using standard kits. The Illumina HiSeq 2500 platform are used to sequence the mutants and the parental strain on one lane using Rapid Run mode that would result in 9-10 million reads per sample. Mapping and Assembly with Qualities software are utilized to map the reads produced by the Illumina sequencer to the reference genome (parental reference strain). Individual high-confidence SNPs are identified. Genetic variant annotation and effect prediction software (snpEff and snpSift) are utilized to predict the impact of a specific mutation on protein function^{74,75}. Select SNPs are confirmed independently by PCR amplifying and sequencing of the PCR product. Potential target proteins, other than CA, are expressed and purified using affinity-tag purification to allow for follow-up assays.

In Vivo Pharmacokinetics:

Valuable insight is gained in analyzing the comparative pharmacokinetics of oral and injectable CAI analogues. The data help us determine the dose needed to achieve certain therapeutic concentration (>MIC) of each analog and how frequently doses must be administered to maintain therapeutic concentration (therapeutic time >20 h is optimal for complete eradication of an uncomplicated gonococcal urogenital tract infection) for an optimal clinical response^{86,87}. Also, this will help us identify molecules with low absorption that could be used by injection instead of oral administration. We do not anticipate any serious bioavailability issues with these drugs for systemic application given the bioavailability of CAIs.

Briefly, prioritized CAI analogs (4-8 analogs) are administered either intravenously (i.v.) or orally (p.o) at doses ranging from 3 mg/kg-200 mg/kg. These are projected doses and are subject to modification after the initial test. The drugs are administered into 10-12 week old BALB/c male and female mice. Blood samples and urine are collected at 0, 0.25, 0.5, 1, 2, 4, 8, 12 and 24 hours post-injection (n=4 animals per time point). Using the analytical method developed for each compound, drugs are quantified by internal standardization, liquid-liquid extraction, and HPLC-MS/MS analysis (API 5500 QTrap) in the Purdue Translational Pharmacology Core Facility (see letter of support from Dr. Greg Knipp) Common PK and ADMET parameters routinely evaluated by the core that are collected include area under the curve (AUC), area under the moment curve (AUMC), drug half-life (t_{1/2}), maximum plasma concentration (Cₘₐₓ) and time (tₘₐₓ) to reach Cₘₐₓ. Bioavailability (F) are estimated by comparing AUCs of i.v. and p.o. for each molecule. From the data collected, we will calculate the pharmacological indices common to antibiotic therapy⁸⁸: 1) cumulative percentage of time the free drug concentration is above the MIC (ƒT_{>MIC}), 2) the ratio of the area under the free-drug concentration time curve to the MIC (ƒ AUC/MIC), and 3) the ratio of unbound peak plasma drug concentration to the MIC (ƒ Cₘₐₓ/MIC). These PK/pharmacodynamic (PD) indices will accurately provide metrics to assess predicted antibiotic efficacy for the proposed in vivo efficacy models below. Our goal is to select for compounds that maintain plasma concentrations 4 times the in vitro MIC₅₀ with therapeutic time of >20 h for an optimal clinical response^{86,87,89}.

Evaluate In Vivo Safety Profile and Toxicity

The maximum tolerated dose (MTD) in mice is calculated and is expected to be several-fold higher than therapeutic dose based on the safety profile of CAIs. The MTD studies are performed in CFW mice in 2 phases. In Phase A, the dose level are increased until the MTD is determined (n=6 animals/dose using 3 males and 3 females). The MTD is a dose that produces neither mortality nor more than a 10% decrement in body weight nor clinical signs of toxicity or a significant change in renal and/or hepatic function in the survivors. In Phase B, animals are dosed daily for 7 days at fractions of the single dose MTD to estimate a repeat dose MTD (n=10 animals/dose, 5 males and 5 females, with control, low, mid, and high dose determined from the phase A studies). During each phase mice are observed daily for body weight gain and clinical signs of abnormality, renal and hepatic function. At termination of Phase B, samples for clinical chemistry, hematology, and histopathology are collected. This data are used to calculate the therapeutic window for analogs tested.

In Vivo Testing in the Female N. gonorrhoeae Mouse Model

Although N. gonorrhoeae is a strict human pathogen, estradiol-treated mice can be infected with N. gonorrhoeae and several aspects of murine infection in this model mimic natural gonococcal infection in humans⁹⁰⁻⁹². This model has been successfully utilized to evaluate the efficacy of various antimicrobials against N. gonorrhoeae^{86,93,94}. Briefly, 8-10 weeks old female BALB/c mice in the diestrus stages of the estrous cycle (identified by cytological examination of vaginal smears) are intradermally implanted with a 5-mg, 21-day-slow-release 17β-estradiol pellet. To reduce the overgrowth of commensal flora that occurs under the influence of estradiol and to increase susceptibility to N. gonorrhoeae, mice are treated with an antibiotic cocktail (1.2 mg/mouse streptomycin and 0.6 mg/mouse vancomycin twice daily by intraperitoneally injection in addition to 0.04 g/100 ml of trimethoprim in drinking water). Two days after pellet implantation (day 0), mice are anesthetized and the vagina are rinsed with 30 μl of 50 mM HEPES (pH 7.4) followed by intravaginal inoculation with 20 μl (˜4×10⁶ CFU strain ATCC 700825) gonococci suspension in PBS containing 0.5 mM CaCl₂) and 1 mM MgCl₂⁹⁵. Two days post infection, each group of mice is treated with different doses of AZM analogs (dose is determined from PK studies) or control vehicle for 5 days (this is an initial time point to get an idea about bacterial burden and dose and can may be modified later). Groups of mice will receive control antibiotics ciprofloxacin (12.5 mg/kg) or ceftriaxone (15 mg/kg) as a positive control⁹⁴. We envision testing up to 5 new analogs and two additional FDA-approved CAIs (AZM and EZM). Pre- and posttreatment cultures are performed by collecting vaginal mucus from all mice with a moistened sterile swab and suspending the swab contents in 1 ml of GC broth (GCB). Undiluted and diluted samples are cultured for N. gonorrhoeae on GC agar with antibiotics. Mice are considered to have cleared the infection when cultures of vaginal swab specimens are negative (no CFU recovered) for three or more consecutive days. Additional experiments are designed as needed to identify dose/frequency/time needed for complete clearance of N. gonorrhoeae from mice once potent inhibitors are identified

Sex as a Biological Variable:

Male and female animals are used in approximately equal numbers in each experiment (PK and toxicity) except for the N. gonorrhoeae vaginal infection model where only females are used. Data are initially analyzed irrespective of sex but also the data are separately analyzed based on sex, to determine if there is any differential response to compound administration.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

What is claimed is:

1. A method for treating a patient with a bacterial infection comprising the step of administrating therapeutically effective amount of a carbonic anhydrase inhibitor or a pharmaceutically acceptable salt thereof, together with one or more diluents, excipients or carriers, to the patient in need of treatment for said infection.

2. The method according to claim 1 further comprising one or more other antibiotics.

3. The method for treating a patient with a bacterial infection according to claim 1, wherein said bacterial is Neisseria gonorrhoeae.

4. The method for treating a patient with a bacterial infection according to claim 1, wherein said bacterial infection is a sexually transmitted disease.

5. The method of claim 1, wherein said carbonic anhydrase inhibitor has the formula 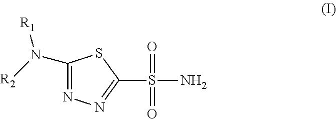
or a pharmaceutically acceptable salt thereof, wherein
R₁ is hydrogen, an acyl, alkyl, alkenyl, alkynyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted; and
R₂ is hydrogen, an acyl, alkyl, alkenyl, alkynyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted.

6. The method of claim 5, wherein said carbonic anhydrase inhibitor is

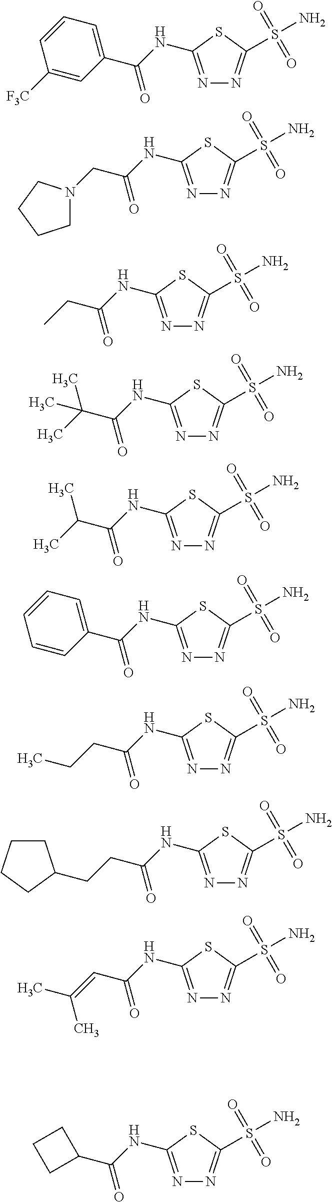

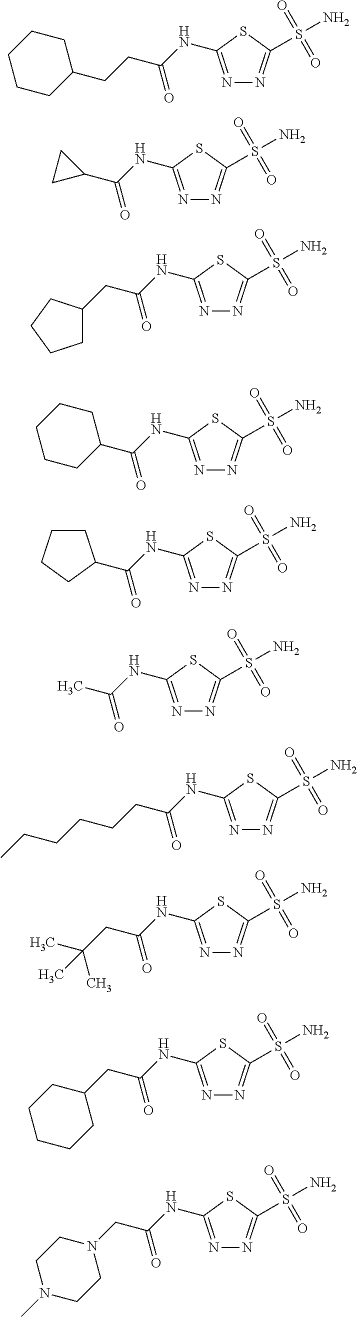

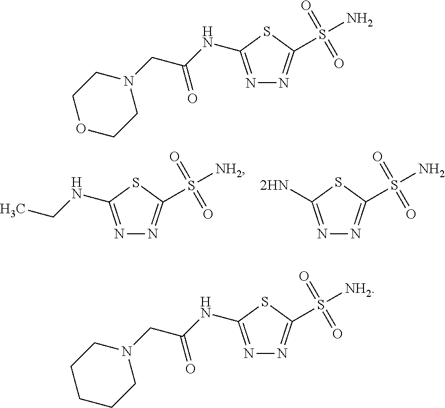

7. A carbonic anhydrase inhibitor compound having the formula 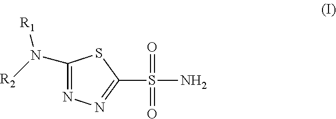
or a pharmaceutically acceptable salt thereof, wherein
R₁ is hydrogen, an acyl, alkyl, alkenyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted; and
R₂ is hydrogen, an acyl, alkyl, alkenyl, alkynyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted.

8. The compound of claim 7, wherein said compound is

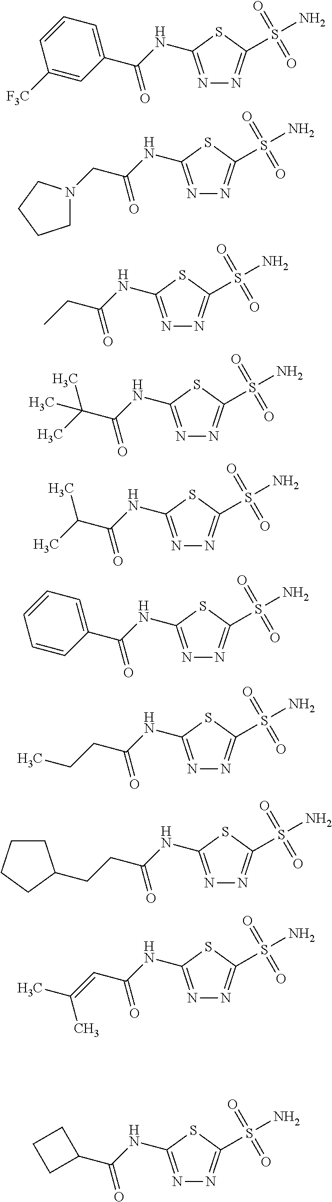

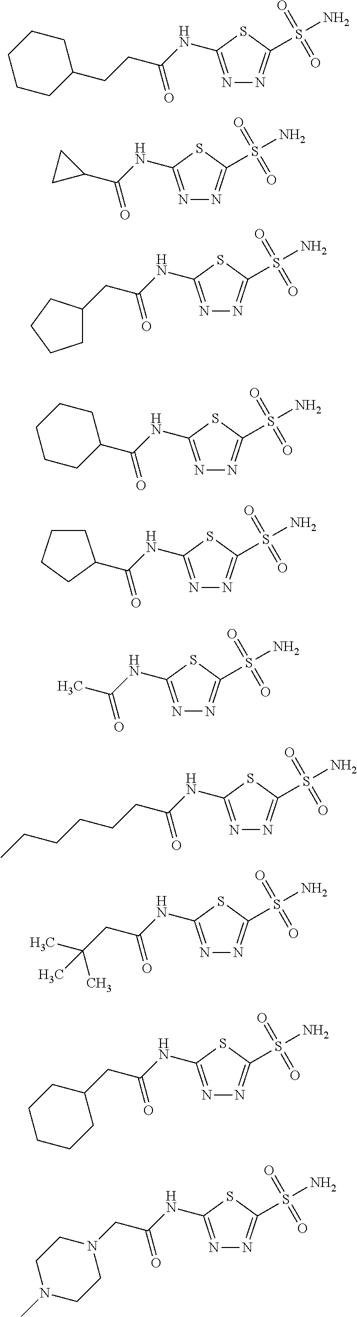

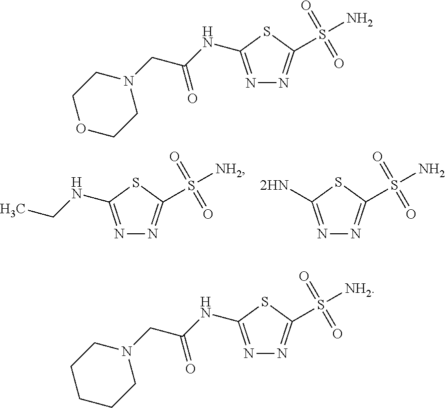

9. A pharmaceutical composition comprising one or more compounds of formula (I) according to claim 7, or a pharmaceutically acceptable salt thereof, together with one or more diluents, excipients or carriers.

10. A pharmaceutical composition comprising one or more compounds of formula (I) according to claim 7, or a pharmaceutically acceptable salt thereof, together with one or more diluents, excipients or carriers, for use in the treatment of a bacterial infection.

11. A pharmaceutical composition comprising one or more compounds of formula (I) according to claim 7, or a pharmaceutically acceptable salt thereof, together with one or more other antibiotics, and one or more diluents, excipients or carriers, for use in the treatment of a bacterial infection.

12. A method for treating a patient with a bacterial infection comprising the step of administrating therapeutically effective amount of a carbonic anhydrase inhibitor or a pharmaceutically acceptable salt thereof, in combination with one or more other compounds of the same or different mode of action, together with one or more diluents, excipients or carriers, to the patient in need of treatment for said infection.

13. The method for treating a patient with a bacterial infection according to claim 12, wherein said bacterial is Neisseria gonorrhea.

14. The method for treating a patient with a bacterial infection according to claim 12, wherein said bacterial infection is a sexually transmitted disease.

15. The method of claim 12, wherein said carbonic anhydrase inhibitor has the formula 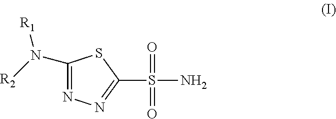
or a pharmaceutically acceptable salt thereof, wherein
R₁ is hydrogen, an acyl, alkyl, alkenyl, alkynyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted; and
R₂ is hydrogen, an acyl, alkyl, alkenyl, alkynyl, heteroalkyl, heteroalkenyl, heterocyclyl, cycloalkyl, cycloalkenyl, cycloheteroalkyl, cycloheteroalkenyl, alkylaryl, alkenylaryl, aryl, arylalkyl, and arylalkenyl, each of which is optionally substituted.

16. The method of claim 15, wherein said carbonic anhydrase inhibitor is 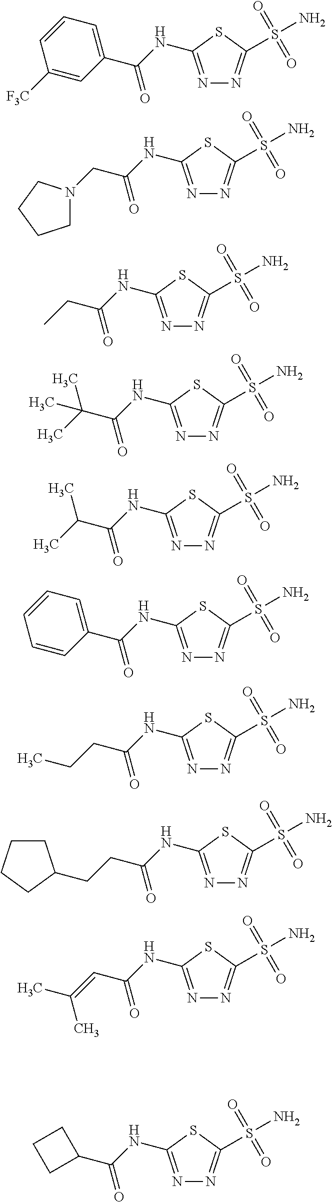 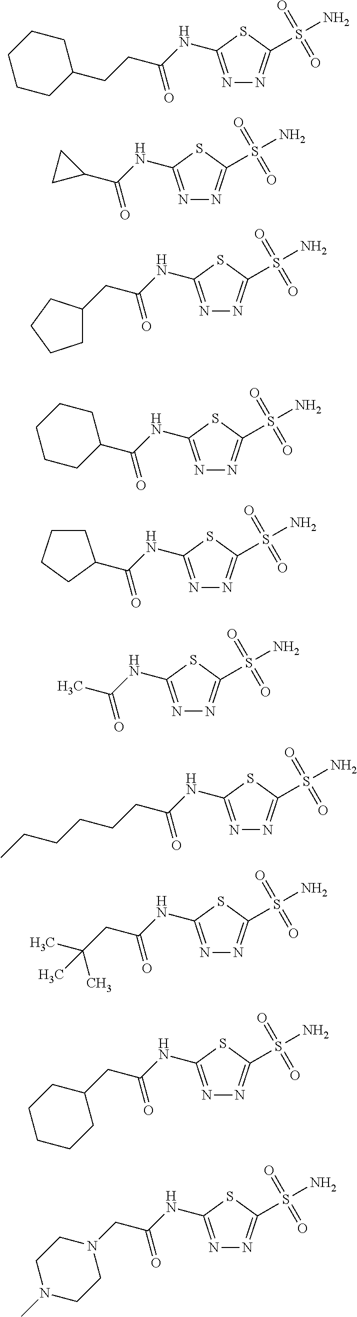 [chemical structure]
